(19) United States
(12) Patent Application Publication
(10) Pub. No.: US 2010/0031734 A1
(43) Pub. Date: Feb. 11, 2010

(54) METHOD AND SYSTEM FOR DETECTING IMPURITIES IN LIQUIDS

(75) Inventors: BIN ZHANG, VISTA, CA (US); TOSHITAKA NAKAMURA, OCEANSIDE, CA (US); AMANE MOCHIZUKI, SAN DIEGO, CA (US)

Correspondence Address: KNOBBE MARTENS OLSON & BEAR LLP, 2040 MAIN STREET, FOURTEENTH FLOOR, IRVINE, CA, 92614, US

(73) Assignee: NITTO DENKO CORPORATION, OSAKA (JP)

(21) Appl. No.: 12/186,452

(22) Filed: Aug. 5, 2008

(51) Int. Cl. G01N 33/00

(52) U.S. Cl. 73/61.43

(57) ABSTRACT

A method for testing the quality of a solvent is disclosed. The method comprises obtaining a solvent sample, wherein the solvent sample contains less than 10 ppm of impurities and nebulizing the solvent sample thereby forming a multitude of droplets that comprises solvent and impurities. The method further comprises evaporating the solvent from at least a portion of the multitude of droplets to thereby form a multitude of aerosol particles, condensing a liquid onto at least a portion of the multitude of aerosol particles to a multitude of form liquid-coated aerosol particles, and counting the number of liquid-coated aerosol particles.

### BACKGROUND

1. Field of the Invention

This invention relates to detection, quantification and monitoring of nanoparticles in liquid solution or suspension. Particularly, the invention relates to methods and systems for detecting nanoparticles, monitoring of liquid quality, and for use in manufacturing quality control of purification treatments.

2. Description of the Related Art

Measurement of impurities in liquids is important for many industries because there are often requirements for certain levels of purities in liquids used for various purposes. For example, a high level of purity may be necessary for water or solvents used in research or industry while some minerals may be acceptable or even desirable in drinking water. For the industry that produces pure or ultrapure liquids, detection and measurement of minute amount of impurities is also needed for quality control purposes. The effectiveness of a purification treatment and apparatus may also be assessed by measuring the amount of impurities in the downstream liquid at different times.

The impurities in liquids may come from their natural origins or exist as artificial additives, and may be present as solid particles or dissolved minerals or salts. The conventional methods for detecting impurities in liquids include dynamic light scattering (DLS) for solid particles and electrical conductivity (EC) measurement for dissolved minerals or salts. However, there are limitations for these methods. The DLS has low detection efficiency for very small particles (e.g. <50 nm) at relatively low concentration and for small particles at very low concentrations. The scattered light intensity is too weak in these situations to allow effective detection and counting. While the need for higher purity and better control of particle sizes in liquids has become more prominent with advancement of technologies and biological sciences, the DLS has become less effective for these applications. For example, ultra-pure water is indispensable to the manufacture of IC semiconductors. In particular, since the pattern size of current ultra-large scale integrations (ULSIs) is as small as 100 nm, ultrapure water must not contain impurities with particle sizes of 10 nm or above. Furthermore, DLS may not be able to detect solid particulates that are transparent to the incoming light such as some bacteria. As for the EC measurement, it is not effective in detecting weakly charged or neutral dissolved salts. A better method and system for detecting very small particles (e,g., in the range of less than 50 nm) and salts and/or detecting very low concentration of impurities in a liquid are desirable and would satisfy a long-felt need for various applications.

### SUMMARY

An embodiment provides a method for testing the quality of a solvent, comprising obtaining a solvent sample, wherein the solvent sample contains less than 10 ppm of impurities, nebulizing the solvent sample thereby forming a multitude of droplets, the droplets comprising solvent and impurities, evaporating the solvent from at least a portion of the multitude of droplets to thereby form a multitude of aerosol particles, wherein the aerosol particles comprise at least a portion of the impurities, condensing a liquid onto at least a portion of the multitude of aerosol particles to a multitude of form liquid-coated aerosol particles, and counting the number of liquid-coated aerosol particles.

Another embodiment provides a method for monitoring the effectiveness of a purification treatment comprising subjecting a liquid to the purification treatment to thereby form a purified liquid, collecting a liquid sample from the purified liquid, forming droplets from the liquid sample, wherein the droplets comprise residual impurities, evaporating liquid from the droplets to thereby form aerosol particles comprising at least a portion of the residual impurities, condensing a second liquid onto the aerosol particles, counting the number of aerosol particles, and determining the effectiveness of the purification treatment by evaluating the number of aerosol particles counted.

Another embodiment provides a system for detecting impurities in a liquid sample comprising a nebulizer configured for producing droplets from the liquid sample, an evaporator configured for isolating or forming a plurality of nanoparticles comprising the impurities, a charger configured to charge the plurality of nanoparticles, a differential mobility analyzer (DMA) configured for separating the plurality of nanoparticles according to size, and a condensation particle counter (CPC) configured for detecting the nanoparticles received from DMA.

These and other embodiments are described in greater detail below.

### BRIEF DESCRIPTION OF THE DRAWINGS

FIG. 1 shows a block diagram of a system used for detecting impurities in a liquid sample.

FIG. 2 shows particle size distributions of several types of water as measured by DLS.

FIG. 3 shows particle size distributions of several types of water as measured by the exemplified system for detecting impurities in liquid.

FIG. 4 shows particle size distributions of pure water and water containing polystyrene latex (PSL) particles at a very dilute concentration and a moderate concentration.

FIG. 5 shows the particle counts of pure water and water containing very dilute amount of PSL particles.

### DETAILED DESCRIPTION OF THE EMBODIMENTS

The disclosed embodiments are useful for detecting impurities that have small particle sizes and/or exist in a low concentration in various liquids. The impurities may comprise suspended solids and/or dissolved solids, including salts and minerals, and may be naturally existing or artificially added to the liquid. These embodiments are especially useful for quality control purposes. The description below provides examples where the disclosed system and method can be useful while traditional DLS does not work properly or effectively. For example, the purity and the mineral or salt concentration of drinking water can easily be monitored or tested. The effectiveness of a filter or a filtration system can be evaluated by analyzing the treated liquid. Furthermore, the quality of the water and/or solvents used for the semiconductor industry can also be tested prior to downstream processing. While the DLS is capable of detecting and analyzing particles with a broad distribution of size in liquid, it lacks the ability to detect small particles and dissolved salts or minerals. On the other hand, various embodiments excel in detecting and analyzing particles with sizes in the range of about 4 nm to about 200 nm.

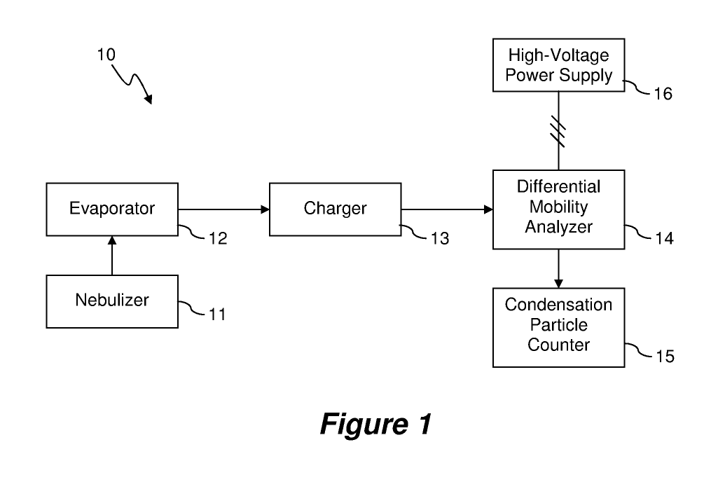

One embodiment provides a system for detecting impurities in a liquid sample. As depicted in FIG. 1, the illustrated system 10 comprises a nebulizer 11 configured for producing droplets from the liquid sample. The nebulizer 11 is configured so that micron sized liquid droplets can be produced by passing a relatively high pressure carrier gas through the liquid sample (detail not illustrated in FIG. 1). In some embodiments, the carrier gas comprises nitrogen, helium, air or any other gas (or mixture thereof) that does not result in any significant negative effect on the operation of the nebulizer and does not adversely affect the downstream measurements. Suitable nebulizers are available from commercial sources.

The system 10 also comprises an evaporator 12 configured for isolating or forming a plurality of particles or nanoparticles comprising the impurities. The evaporator 12 is capable of removing or drying the liquid in the droplets produced by the nebulizer 11. In some embodiments, the insoluble solids in the droplets may be isolated, thereby forming a plurality of nanoparticles as the liquid is removed from the droplets. In some embodiments, the dissolved (soluble) salts or minerals may precipitate out to form a plurality of nanoparticles when the liquid in the droplets are dried. In some embodiments, the nanoparticles may comprise the impurities. In an embodiment, the nanoparticles comprise both soluble and insoluble impurities. In some embodiments, the impurities comprise dissolved solids and/or suspended solids. In some embodiments, the nanoparticles comprise particles having sizes in a range of about 4 nm to about 80 nm or about 4 nm to about 50 nm. Suitable evaporators are available from commercial sources.

In one embodiment, the system 10 optionally comprises a charger 13 and a differential mobility analyzer (DMA) 14. The charger 13 is configured to charge the plurality of nanoparticles received from the evaporator 12 with an ionizing unit. The charger 13 may be a bipolar ion charger. Suitable chargers are available from commercial sources. In some embodiments, the nanoparticles may be charged by gamma (γ) radiation. The DMA 14 is configured for separating the plurality of charged nanoparticles received from the charger 13 according to size. Suitable DMA's are available from commercial sources. The center electrode of the DMA 14 is biased with a high voltage (e.g., up to 10,000 VDC) supplied by a high-voltage power supply 16. Depending on the aerosol flow rate received from the charger 13 and the applied voltage, a balance between electrical and drag forces defines a unique particle trajectory for each mobility degree, and only particles within a narrow range of electrical mobility have the correct trajectory to pass through an open slit near the exit of the DMA 14 at a time. By sweeping the center rod voltage in the DMA 14, particles would exit the DMA in waves of different narrow range of sizes.

The system 10 further comprises a condensation particle counter (CPC) 15. The CPC 15 is configured for detecting the aerosol particles received from the evaporator 12 or for detecting the size-categorized nanoparticles received from the DMA 14. The CPC 15 condenses a liquid onto at least a portion of the multitude of aerosol particles to form a multitude of liquid-coated aerosol particles and count the number of liquid-coated particles. In some embodiments, when the DMA 14 is combined with the CPC 15, a particle size distribution of a plurality of nanoparticles can be measured. Suitable CPC's are available from commercial sources.

An embodiment provides a method for testing the quality of a solvent. In some embodiments, the solvent may be water, such as drinking water, purified water, ultrapure water, de-ionized water, biological level water, or microelectronic level water. In some embodiments, the solvent may be a solvent for use in a laboratory or in an industrial process, such as industrial solvent or reaction/reagent grade solvent. “Ultrapure” water (Type I) has been described as the grade required for critical laboratory applications such as high performance liquid chromatography (HPLC) mobile phase preparation, blanks and sample dilution in gas chromatography (GC), HPLC, atomic absorption (AA) spectroscopy, inductively coupled plasma mass spectrometry (ICP-MS) and other advanced analytical techniques, preparation of buffers and culture media for mammalian cell culture and in vitro fertilization; production of reagents for molecular biology applications (e.g., DNA sequencing and polymerase chain reaction), and preparation of solutions for electrophoresis and blotting. “Pure” (Type II) water has been described as the grade used in general laboratory applications such as buffers, pH solutions and microbiological culture media preparation; as feed to Type I water systems, clinical analyzers, cell culture incubators and weatherometers; and for preparation of reagents for chemical analysis or synthesis. Different published norms define the quality required for specific laboratory water applications: ASTM® and ISO® 3696 for laboratory applications; CLSI guidelines for clinical laboratories. Some laboratories also use norms defined in the European or the US Pharmacopoeia.

In some embodiments, the solvent may contain impurities at a level of less than about 500, 400, 300, 200, 100, 50, 10, 5, 3 or 2 ppm. In some embodiments, the impurities may comprise at least one kind of insoluble solid, such as dust particles, suspended particles, insoluble organic matter or insoluble inorganic matter. In some embodiments, the impurities may comprise at least one type of soluble or dissolved solid, such as soluble salts or minerals, dissolved metal or metalloids, or dissolved organics. The impurities may be either organic or inorganic, and may be artificially added and/or naturally exist in the solvent.

In an embodiment, the method comprises obtaining a solvent sample and nebulizing the solvent sample to thereby forming a multitude of droplets. For example, nebulizing the solvent sample may comprise passing a relatively high pressure carrier gas (e.g., nitrogen, helium, air, or combination thereof) through a nebulizer 11 to produce a plurality of micron sized liquid droplets. The nebulized droplets may comprise a multitude of droplets having sizes in the range of about 1 μm to about 200 μm, about 1 μm to about 150 μm, about 1 μm to about 100 μm, or about 1 μm to about 50 μm. In some embodiments, at least about 95%, about 90%, about 85% or about 80% of the multitude of droplets have sizes in a range of about 1 μm to about 200 μm, about 1 μm to about 150 μm, about 1 μm to about 100 μm or about 1 μm to about 50 μm. In some embodiments, the nebulized droplets may comprise solvent and some impurities. In other embodiments, the nebulized droplets may comprise solvent without the presence of any detectable impurity.

The method may further comprise evaporating the solvent from at least a portion of the multitude of droplets, thereby forming a multitude of aerosol particles. The aerosol particles comprise solid particles suspended in a carrier gas downstream of the evaporation stage. In some embodiments, the aerosol particles comprise at least a portion of the impurities. In some embodiments, the dissolved or soluble impurities may precipitate out as solid particles while the solvent is being evaporated from the droplets. In some embodiments, the insoluble solid(s) are left behind as solid particles when the solvent in the droplet is evaporated. In some embodiments, the solid particles may comprise particles of different shape, clusters or agglomerates. In some embodiments, the multitude of aerosol particles has sizes in a range of about 4 nm to about 200 nm, about 4 nm to about 150 nm, about 4 nm to about 100 nm, or about 4 nm to about 80 nm. In some embodiments, at least about 95%, about 90%, about 85% or about 80% of the multitude of aerosol particles have sizes in a range of about 4 nm to about 200 nm, about 4 nm to about 150 nm, about 4 nm to about 100 nm, or about 4 nm to about 80 nm.

Optionally in some embodiments, the method also comprises separating the multitude of aerosol particles according to particle size to form a multitude of size-categorized aerosol particles. In an embodiment, the aerosol particles may first be charged by passing through the charger 13, which can bring aerosols to a Fuchs equilibrium charge distribution. The charged aerosol particles can then enter an annular column in the DMA 14 to be separated according to size. As different size aerosol particles exit the DMA 14, a multitude of size-categorized aerosol particles are formed.

The method then proceeds by condensing a liquid onto at least a portion of the multitude of aerosol particles or size-categorized aerosol particles to form a multitude of liquid-coated aerosol particles or size-categorized aerosol particles, and then proceeds by counting the number of liquid-coated aerosol particles or size-categorized aerosol particles. In some embodiments, the aerosol particles enter the CPC 15 directly from the evaporator 12. In some embodiments, the size-categorized aerosol particles enter the CPC 15 after exiting the DMA 14. The aerosol particles or the size-categorized aerosol particles may first pass through a heated saturator where a liquid with high vapor pressure (e.g., butanol for one type of CPC) is vaporized and diff-used into the main stream (not illustrated in FIG. 1). The mixture of the aerosol particles or the size-categorized aerosol particles and liquid vapor may then enter a cooled condenser where the liquid vapor becomes supersaturated and condenses around the aerosol particles or the size-categorized aerosol particles by using the particles as condensation nuclei. The originally small particles quickly grow into larger particles after condensation. These enlarged particles then can be more easily counted by a conventional optical detector. In some embodiments, by sweeping the center rod voltage in the DMA 14 in combination with the use of the CPC 15, a particle size distribution can be measured.

Another embodiment provides a method for monitoring the effectiveness of a purification treatment. In some embodiments, the purification treatment may be selected from clarification, water conditioning, ion exchange, filtration, sedimentation and distillation. The method begins by subjecting a liquid to the purification treatment to thereby form a purified liquid. In some embodiments, the liquid may be water or a solvent for use in a laboratory or in an industrial process.

The method continues to the steps of collecting a liquid sample from the purified liquid and forming droplets comprising residual impurities from the liquid sample. In an embodiment, the residual impurities comprise an insoluble solid. In some embodiments, the droplets are formed by introducing the liquid sample into the nebulizer 11. The droplets then enter the evaporator 12 for evaporating liquid from the droplets to thereby form aerosol particles comprising at least a portion of the residual impurities. In some embodiments, the droplets have sizes in a range of about 1 μm to about 200 μm, about 1 μm to about 150 μm, about 1 μm to about 100 μm or about 1 μm to about 50 μm. In some embodiments, at least about 95%, about 90%, about 85% or about 80% of the multitude of droplets have sizes in a range of about 1 μm to about 200 μm, about 1 μm to about 150 μm, about 1 μm to about 100 μm or about 1 μm to about 50 μm. In some embodiments, the aerosol particles have sizes in a range of about 4 nm to about 200 nm, about 4 nm to about 150 nm, about 4 nm to about 100 nm, or about 4 nm to about 80 nm. In some embodiments, at least about 95%, about 90%, about 85% or about 80% of the the aerosol particles have sizes in a range of about 4 nm to about 200 nm, about 4 nm to about 150 nm, about 4 nm to about 100 nm, or about 4 nm to about 80 nm.

Next the aerosol particles are separated according to particle size. In some embodiments, the aerosol particles are first charged by the charger 13 for the separation by particle size in the DMA 14. Once the aerosol particles are separated, they proceed to enter the CPC 15, where a second liquid is condensed onto the aerosol particles. The particular second liquid depends upon the type of CPC used. In some embodiments, the second liquid may be butanol, in others, water, depending upon the type or series of CPC used. The number of aerosol particles may be counted by an optical detector.

In an embodiment, the method further comprises determining the effectiveness of the purification treatment by evaluating the number of aerosol particles counted. In some embodiments, determining the effectiveness of the purification treatment comprises repeating the method described above to thereby obtain a second number of aerosol particles, and then comparing the second number of aerosol particles to the number of aerosol particle counted.

The above-described embodiments typically have high detection efficiency for nanoparticles or particles smaller than about 200 nm, especially for particles that have particle sizes that are less than about 50 nm, even at relatively low concentrations. These embodiments also work well for detecting very diluted liquids, such as those that have very low concentration and small particles.

Results from a number of experiments conducted according to the method and system of the present invention are described below. These experiments are intended for illustrative purposes only and are not intended to limit the scope of the present invention.

### EXAMPLE 1

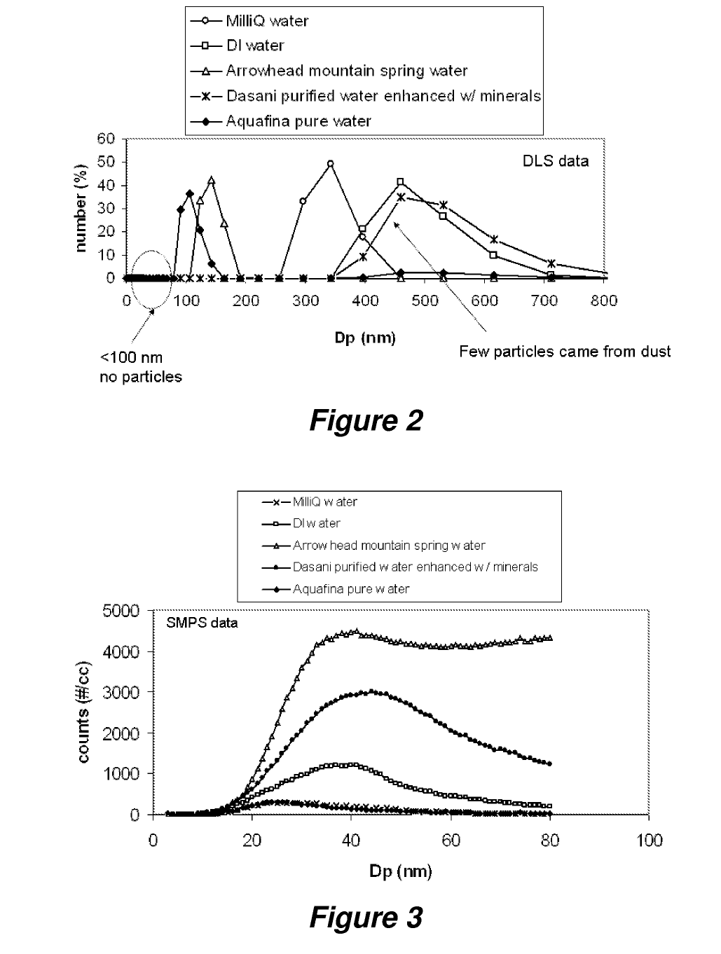

Several types of water samples were tested for their impurity level by both DLS and the exemplified method describe above. The tested water samples include lab level water such as de-ionized water (“DI water”), tap water filtered through a 0.2 μm MilliO filter (“MilliQ water,” Millipore Inc., Billerica, Mass.), and some commercially available drinking water samples such as Arrowhead® mountain spring water (“Arrowhead water,” Nestlé Waters North America Inc., Wilkes Barre, Pa.), Dasani® purified water enhanced with minerals (“Dasani water,” Coca-Cola Company, Atlanta, Ga.), and Aquafina® pure water (“Aquafina water,” PepsiCo Inc., Wichita, Kans.). First, the impurity levels of all the water samples were measured by DLS (model Nano-S, Malvern Inc., Westborough, Mass.). As shown in FIG. 2, the DLS measurements did not detect any particles smaller than about 100 nm in any of the water samples. In addition, the DLS measurements were unable to distinguish them very well. The larger particles detected are believed to be dust particles in the water.

Then the measurements were conducted for the same samples by the exemplified method, and the size distribution of each sample was obtained. As shown in FIG. 3, different size distributions in the size range of about 4 nm to about 80 nm were observed when the current method was used. Since each sample appears to be clear, it is likely that the detected particles were from dissolved minerals. Among the water samples tested, MilliQ water and Aquafina water have the lowest and similar impurity level, while Arrowhead water shows highest population of particles. The particle concentrations from Dasani water and DI water sit in the middle level of impurity, in which DI water indicated a lower particle concentration compared with Dasani water. The data clearly shows the capability of the exemplified system and method to distinguish pure water samples containing very small particles or dissolved minerals. The current method and system can also detect dissolved minerals.

### EXAMPLE 2

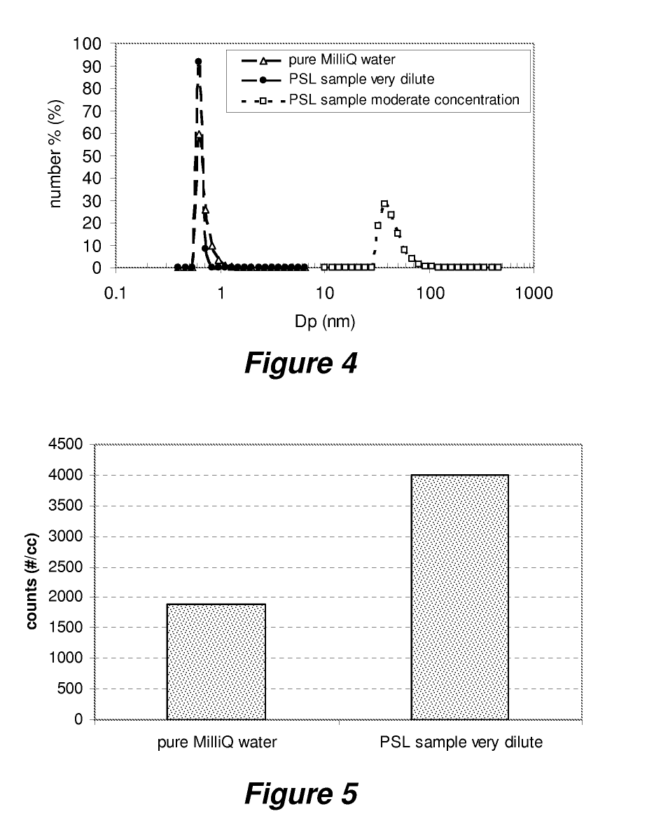

To verify the capability of the exemplified system and method to measure particles in very dilute sample when DLS can not be effectively utilized, a comparison experiment was conducted. An amount of 40 nm polystyrene latex (PSL) particles from Duke Scientific Corp. (Fremont, Calif.) were introduced into MilliQ water, which has been shown to contain the least amount of impurity among the water samples used in Example 1. The PSL suspended water sample was serially diluted multiple times by adding MilliQ water until the DLS measurement could not reasonably detect impurities. FIG. 4 is a plot of number distribution vs. particle size measured by DLS. The peak at less than 1 nm is believed to be due to the system error of DLS. As concluded from Example 1, DLS can not detect any particles in the MilliQ water. When one of the PSL suspended samples with moderate PSL concentration was tested by DLS, a well shaped distribution with a peak at ˜40 nm was seen in FIG. 4.

Once the PSL sample has been serially diluted about 10 times, a distribution similar to pure MilliQ water was obtained and no particles were detected by DLS. At this dilution level (a dilution factor of about 1024), which correspond to PSL concentration of about 3 ppm, there were so few 40 nm PSL particles suspended in the water that they could not scatter enough incoming light for DLS to detect them. As a result, the produced light signal in DLS was too weak to be effectively recognized. The same PSL samples were also measured by the exemplified system. At a moderate PSL concentration, the size distribution obtained by the exemplified system had a peak at ˜40 nm and was similar to the DLS measurement. For the very dilute sample, the PSL concentration was also too low that no reasonable size distribution could be measured by the exemplified system. However, during this measurement, it is possible that most of the PSL particles were trapped in the pipes during delivery to the CPC (TSI Inc., Shoreview, Minn.). At such low concentration, the portion of trapped particles during delivery became dominant. To prove this, the PSL particles were introduced directly into the CPC from the evaporator instead of first passing through the charger and DMA. This configuration would not provide a size distribution, but a total particle concentration could still be obtained by CPC measurements. As shown in FIG. 5, for pure MilliQ water as a background, CPC detected a total concentration of ˜1900 #/cc, while for this very dilute PSL particle sample, CPC showed a total concentration of ˜4000 #/cc. This demonstrated that the exemplified system can identify the existence of particles in very dilute sample where DLS does not function well.

What is claimed is:

1. A method for testing the quality of a solvent comprising:
obtaining a solvent sample, wherein the solvent sample contains less than 10 ppm of impurities;
nebulizing the solvent sample thereby forming a multitude of droplets, the droplets comprising solvent and impurities;
evaporating the solvent from at least a portion of the multitude of droplets to thereby form a multitude of aerosol particles, wherein the aerosol particles comprise at least a portion of the impurities;
condensing a liquid onto at least a portion of the multitude of aerosol particles to form a multitude of liquid-coated aerosol particles; and
counting the number of liquid-coated aerosol particles.

2. The method of claim 1, further comprising:
charging at least a portion of the multitude of aerosol particles; and
separating the multitude of aerosol particles according to particle size to form a multitude of size-categorized aerosol particles.

3. The method of claim 1, wherein the impurities comprise soluble impurities.

4. The method of claim 3, further comprising precipitating at least a portion of the soluble impurities to thereby form a multitude of aerosol particles.

5. The method of claim 1, wherein the multitude of aerosol particles have sizes in a range of about 4 nm to about 200 nm.

6. The system of claim 1, wherein the multitude of droplets have sizes in a range of about 1 μm to about 200 μm.

7. The method of claim 1, wherein the solvent is selected from drinking water, de-ionized water, biological level water, microelectronic level water, industrial solvent, or reagent grade solvent.

8. The method of claim 1, wherein the impurities comprise at least one of insoluble solids, soluble salts and minerals.

9. The method of claim 1, wherein the liquid comprises butanol.

10. A method for monitoring the effectiveness of a purification treatment comprising:
subjecting a liquid to the purification treatment to thereby form a purified liquid;
collecting a liquid sample from the purified liquid;
forming droplets from the liquid sample, wherein the droplets comprise residual impurities;
evaporating liquid from the droplets to thereby form aerosol particles comprising at least a portion of the residual impurities;
condensing a second liquid onto the aerosol particles;
counting the number of aerosol particles; and
determining the effectiveness of the purification treatment by evaluating the number of aerosol particles counted.

11. The method of claim 10, wherein determining the effectiveness of the purification treatment comprises repeating the steps set forth in the method of claim 10 to thereby obtain a second number of aerosol particles; and comparing the second number of aerosol particles to the number of aerosol particle counted.

12. The method of claim 10, wherein the purification treatment is selected from clarification, water conditioning, ion exchange, filtration, sedimentation and distillation.

13. The method of claim 10, further comprising:
charging the aerosol particles; and
separating the aerosol particles according to particle size.

14. The method of claim 10, wherein the aerosol particles have sizes in a range of about 4 nm to about 200 nm.

15. The system of claim 10, wherein the droplets have sizes in a range of about 1 μm to about 200 μm.

16. The method of claim 10, wherein the liquid is water.

17. The method of claim 10, wherein the residual impurities comprise an insoluble solid.

18. The method of claim 10, wherein the second liquid comprises butanol.

19. A system for detecting impurities in a liquid sample comprising:
a nebulizer configured for producing droplets from the liquid sample;
an evaporator configured for isolating or forming a plurality of nanoparticles comprising the impurities;
a charger configured to charge the plurality of nanoparticles;
a differential mobility analyzer (DMA) configured for separating the plurality of nanoparticles according to size; and
a condensation particle counter (CPC) configured for detecting the nanoparticles received from DMA.

20. The system of claim 19, wherein the liquid sample comprises pure or ultrapure water.

21. The system of claim 19, wherein the liquid sample comprises de-ionized water, biological level water, or microelectronic level water.

22. The system of claim 19, wherein the impurities comprise dissolved solids and/or suspended solids.

23. The system of claim 19, wherein the nanoparticles comprise particles having sizes in a range of about 4 nm to about 80 nm.
